(19) United States
(12) Patent Application Publication
(10) Pub. No.: US 2026/0233218 A1
(43) Pub. Date: Aug. 13, 2026

(54) SEMI-QUANTITATIVE ELECTROCHEMICAL BIOSENSOR SYSTEM AND METHOD FOR PATHOGENIC ORGANISM NUCLEIC ACID DETECTION

(71) Applicant: eDNA Biotech, Inc., Pasadena, CA (US)

(72) Inventor: Di Kang, Glendora, CA (US)

(73) Assignee: eDNA Biotech, Inc., Pasadena, CA (US)

(21) Appl. No.: 19/053,195

(22) Filed: Feb. 13, 2025

(51) Int. Cl. B01L 3/00; B01L 7/00; C12Q 1/48; G01N 27/327

(52) U.S. Cl. CPC B01L 3/502715; B01L 7/52; C12Q 1/485; G01N 27/3277; B01L 2200/16; B01L 2300/0645; B01L 2300/0663; B01L 2300/0819; B01L 2300/087; B01L 2300/1805; G01N 2333/9128

(57) ABSTRACT

A semi-quantitative electrochemical biosensor system employs a benchtop device and disposable test cartridges capable of quickly identifying pathogen DNA at the point of care. The system leverages an electrochemical DNA probe sensor platform in situ with the nucleic acid amplification reaction, which allows for simultaneous semi-quantitative detection of multiple DNA molecules. The cartridge enables in-situ and real time detection of the amplificon using an electrochemical nucleic acid probe sensor during the amplification process. On the cartridge, the electrochemical DNA probe based sensor array is directly integrated in the nucleic acid amplification tests (NAAT) reaction. By placing the eDNA probe sensor in the NAAT reaction, the device can simultaneously measure the reaction produced amplicons while the reaction progresses. The NAAT can be either an isothermal process, or a modified isothermal process with periodic cooling, or a thermal oscillation (e.g. polymerase chain reaction PCR) amplification method.

### BACKGROUND OF THE INVENTION

The present invention relates to semi-quantitative nucleic acid detection systems that utilize in-situ nucleic acid amplification in combination with an electrochemical nucleic acid sensor platform for detection of pathogenic organism nucleic acid.

The standard culture test is time-consuming and causes delays in initiating proper treatment. It takes 48-72 hours to yield results due to the necessary bacterial growth in a controlled environment. Many culture tests are conducted in off-site laboratories, which further extends the time before results are available to the treating physicians. This delay forces clinicians to rely on empirical antibiotic treatments using broad-spectrum antibiotics before the test results become available. This traditional process has significant drawbacks.

Existing molecular tests are limited and overly sensitive. Some labs are adopting advanced molecular tests to speed up the slow turnaround time of cultures. Instead, large panel lab-developed tests are used, which are expensive, not disease-specific, and primarily geared toward bloodstream infections. These tests are typically conducted in reference laboratories, making them less accessible in point-of-care settings. Moreover, these molecular tests are endpoint qualitative, meaning they cannot differentiate between infection and contamination from bacteriuria flora due to their lack of quantitative capability. They can lead to false positives and inappropriate antibiotic use.

The urgency for a solution in diagnosis cannot be overstated. Multidrug-resistant resistance bacterial infections are highly associated with sepsis. The ability to quickly identify the causative bacteria and their resistance profiles is crucial for guiding appropriate antibiotic therapy and improving patient outcomes, which in turn can reduce hospital stays and save millions of dollars in medical costs.

### SUMMARY OF THE INVENTION

Given the significant shortcomings of existing technologies, rapid, accurate, and affordable on-site testing is needed. Embodiments of the present invention provide a system employing a benchtop device and disposable test cartridges capable of quickly identifying bacteria and multidrug-resistant genes at the point of care. This innovative approach leverages the electrochemical DNA (eDNA) probe sensor platform in situ with the nucleic acid amplification method, which allows for the simultaneous semi-quantitative detection of multiple DNA molecules, providing comprehensive diagnostic information quickly and accurately.

The semi-quantitative electrochemical biosensor system and method for pathogenic organism nucleic acid detection according to embodiments of the present invention can overcome the shortcomings of empiric treatment and existing molecular tests, improve patient outcomes and antibiotic stewardship, reduce healthcare costs, and increase the efficiency of healthcare delivery.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a microfluidic electrochemical biosensor cartridge, which includes: a sample loading port; a reagent reservoir containing nucleic acid amplification test (NAAT) reagents, including a target-specific NAAT reaction primer; a nucleic acid amplification reaction chamber (NAAT reaction chamber); an electrochemical sensor array disposed within the NAAT reaction chamber, including an electrochemical sensor configured to detect an amplified target nucleic acid sequence, the electrochemical sensor including an electrode and a plurality of target-specific probe oligonucleotides attached to a surface of the electrode, wherein each probe oligonucleotide is labeled with an electrochemical redox reporter and includes a complementary sequence that is complementary to the target nucleic acid sequence, and configured to undergo conformation changes upon hybridization with the target nucleic acid sequence to change a redox electron transfer rate between the redox reporter and the electrode; a plurality of microchannels fluidically connecting the sample loading port to the reagent reservoir and fluidically connecting the reagent reservoir to the NAAT reaction chamber; and a plurality of cartridge electrical connectors electrically coupled to the electrochemical sensor array.

In some embodiments, the microfluidic electrochemical biosensor cartridge further includes: a substrate, wherein the reagent reservoir, the NAAT reaction chamber, the electrochemical sensor array, and the plurality of microchannels are formed on a first side of the substrate; and a cartridge housing, wherein the substrate is disposed at least partially within the cartridge housing.

In some embodiments, the microfluidic electrochemical biosensor cartridge further includes a sample lysis area disposed on the first side of the substrate, the sample lysis area including one or more sample lysis chambers containing reagents for sample lysis and nucleic acid extraction, wherein the plurality of microchannels fluidically connect the sample loading port to the sample lysis area and fluidic connect the sample lysis area to the reagent reservoir.

In some embodiments, the microfluidic electrochemical biosensor cartridge further includes: one or both of: a first heating element formed on an opposite side of the substrate at a location corresponding to the NAAT reaction chamber and configured to heat the NAAT reaction chamber, and a second heating element formed on the opposite side of the substrate at a location corresponding to the sample lysis area and configured to heat the sample lysis area; and a second plurality of cartridge electrical connectors electrically coupled to the one or both of the first heating element and the second heating element.

In some embodiments of the microfluidic electrochemical biosensor cartridge, the reagent reservoir further contains a reverse transcriptase, and wherein the reagents in the reagent reservoir are lyophilized.

In some embodiments of the microfluidic electrochemical biosensor cartridge, the probe oligonucleotides have a hybridization rate or an annealing temperature with the target nucleic acid sequence that is higher than a hybridization rate or an annealing temperature of the NAAT reaction primer with the target nucleic acid sequence.

In some embodiments of the microfluidic electrochemical biosensor cartridge, the probe oligonucleotides incorporate locked nucleic acids (LNA) or peptide nucleic acids (PNA).

In some embodiments of the microfluidic electrochemical biosensor cartridge, the probe oligonucleotides are single-stranded oligonucleotides, and a 3′ end of each oligonucleotide is modified with the redox reporter or a termination chemical group to prevent extension of the 3′ end.

In some embodiments of the microfluidic electrochemical biosensor cartridge, the probe oligonucleotides are single-stranded oligonucleotides, wherein one end of the oligonucleotides is modified with the redox reporter, and another end of the oligonucleotides is modified with an anchor moieties to attach the oligonucleotides to the electrode surface, by covalent bonding to the electrode, or by forming self-assembled monolayers on the electrode surface, or by chemisorption or adsorption.

In some embodiments of the microfluidic electrochemical biosensor cartridge, the reagent reservoir contains a plurality of target-specific NAAT reaction primers respectively specific to a plurality of target nucleic acid sequences, and the electrochemical sensor array includes a plurality of electrochemical sensors respectively configured to detect amplified ones of the plurality of target nucleic acid sequences.

In another aspect, the present invention provides an electrochemical biosensor system which includes a microfluidic electrochemical biosensor cartridge described above, and further includes an electrochemical detection and control instrument, configured to receive the biosensor cartridge, wherein the instrument includes: a plurality of host electrical connectors configured to be electrically coupled to the plurality of cartridge electrical connectors of the biosensor cartridge; a controller electrically coupled to the plurality of host electrical connectors, configured (a) to transmit and receive electrical signals to and from the biosensor cartridge, including to receive an electrical signal representing the redox electron transfer rate from the biosensor cartridge, and (b) to record and process the electrical signals received from the electrochemical sensor to generate a detection result; and a user interface on an exterior of a housing of the benchtop instrument, electrically coupled to the controller.

In another aspect, the present invention provides an electrochemical biosensor system which includes the microfluidic electrochemical biosensor cartridge described above, and further including an electrochemical detection and control instrument, configured to receive the biosensor cartridge, wherein the instrument includes: a plurality of host electrical connectors configured to be electrically coupled to the plurality of cartridge electrical connectors of the biosensor cartridge; a controller electrically coupled to the plurality of host electrical connectors, configured (a) to transmit and receive electrical signals to and from the biosensor cartridge, including to receive an electrical signal representing the redox electron transfer rate from the biosensor cartridge, and (b) to record and process the electrical signals received from the electrochemical sensor to generate a detection result; and a user interface on an exterior of a housing of the benchtop instrument, electrically coupled to the controller; wherein either: the biosensor cartridge further comprises a first heating element formed on an opposite side of the substrate at a location corresponding to the NAAT reaction chamber to heat the NAAT reaction chamber, or: the electrochemical detection and control instrument includes a second heating element configured to contact an area of the biosensor cartridge corresponding to the NAAT reaction chamber to heat the NAAT reaction chamber.

In some embodiments of the electrochemical biosensor system, the controller is programed to control the first heating element or the second heating element to maintain a temperature of the NAAT reaction chamber at a constant temperature.

In some embodiments of the electrochemical biosensor system, the controller is programed to control the first heating element or the second heating element to: (a) maintain a temperature of the NAAT reaction chamber at a first temperature between 37° C. and 75° C. for a first time duration, (b) then lower the temperature of the NAAT reaction chamber to a second temperature which is 5° C. to 20° C. below the first temperature for a second time duration, the second time duration being 1 to 5 minutes, wherein a cycle including the first time duration followed by the second time duration is 3 to 15 minutes, and (c) repeat the cycle for 1 to 40 times.

In another aspect, the present invention provides a method for detecting a target nucleic acid sequence, which includes: mixing a sample with nucleic acid amplification test (NAAT) reagents, the NAAT reagents including a target-specific NAAT reaction primer; providing the sample mixed with the NAAT reagents to a nucleic acid amplification reaction chamber (NAAT reaction chamber), wherein the NAAT reaction chamber is provided with an electrochemical sensor array disposed therein, including an electrochemical sensor configured to detect amplified ones of the target nucleic acid sequence, the electrochemical sensor including an electrode and a plurality of target-specific probe oligonucleotides attached to a surface of the electrode, wherein each probe oligonucleotide is labeled with an electrochemical redox reporter and includes a complementary sequence that is complementary to the target nucleic acid sequence, and configured to undergo conformation changes upon hybridization with the target nucleic acid sequence to change a redox electron transfer rate between the redox reporter and the electrode; while the NAAT reagents react with the sample to amplify any target nucleic acid sequence in the sample within the NAAT reaction chamber, monitoring an electrical signal generated by the electrochemical sensor array which represents the redox electron transfer rate; and determine whether the target nucleic acid sequence is present in the sample based on the monitored electrical signal.

In some embodiments, the method further includes: while the NAAT reagents react with the sample to amplify any target nucleic acid sequence in the sample within the NAAT reaction chamber, controlling a temperature of the NAAT reaction chamber, including: (a) maintaining a temperature of the NAAT reaction chamber at a first temperature between 37° C. and 75° C. for a first time duration, (b) then lowering the temperature of the NAAT reaction chamber to a second temperature which is 5° C. to 20° C. below the first temperature for a second time duration, the second time duration being 1 to 5 minutes, wherein a cycle including the first time duration followed by the second time duration is 3 to 15 minutes, and (c) repeating the cycle for 1 to 40 times.

In some embodiments of the method, the probe oligonucleotides have a hybridization rate or an annealing temperature with the target nucleic acid sequence that is higher than a hybridization rate or an annealing temperature of the NAAT reaction primer with the target nucleic acid sequence.

In some embodiments of the method, the probe oligonucleotides incorporate locked nucleic acids (LNA) or peptide nucleic acids (PNA).

In some embodiments of the method, the probe oligonucleotides are single-stranded oligonucleotides, and wherein a 3′ end of each oligonucleotide is modified with the redox reporter or a termination chemical group to prevent extension of the 3′ end.

In some embodiments of the method, the probe oligonucleotides are single-stranded oligonucleotides, wherein one end of the oligonucleotides is modified with the redox reporter, and another end of the oligonucleotides is modified with an anchor moieties to attach the oligonucleotides to the electrode surface, by covalent bonding to the electrode, or by forming self-assembled monolayers on the electrode surface, or by chemisorption or adsorption.

It is to be understood that both the foregoing general description and the following detailed description are exemplary and explanatory and are intended to provide further explanation of the invention as claimed.

### BRIEF DESCRIPTION OF DRAWINGS

FIG. 1 illustrates an electrochemical nucleic acid detection system according to an embodiment of the present invention.

FIG. 2 is a functional block diagram of an electrochemical detection and control instrument of the electrochemical nucleic acid detection system.

FIG. 3 schematically illustrates a reaction sensor cartridge of the electrochemical nucleic acid detection system.

FIGS. 4 and 5 illustrate portions of two exemplary reaction sensor cartridges.

FIGS. 6 and 7 show two tables related to materials used in experiments carried out using the electrochemical nucleic acid detection system.

FIG. 8 illustrates portions of two exemplary reaction sensor cartridges used in the experiments.

FIGS. 9A-9C show results of the experiments.

### DETAILED DESCRIPTION OF THE INVENTION

Embodiments of the present invention provide a rapid, sensitive, and specific nucleic acid detection system that utilizes nucleic acid amplification in combination with an electrochemical nucleic acid sensor platform for detection of pathogenic organism nucleic acid. The innovative detection system not only provides a low-cost and less instrument-dependent nucleic acid detection solution but also produces high sensitivity and high specificity results quickly. The system can be easily multiplexed for multiple target nucleic acid sequences at once and can be used as a point-of-care (POC) test and an at-home test.

Embodiments of the present invention are directed to a system that can semi-quantitively detect the target genomic sequence from pathological organisms via a simple amplification process and reliable detection system. The system includes two main components: (a) a multifunction electrochemical detection and control instrument; (b) a reaction sensor cartridge which extracts pathogen DNA (deoxyribonucleic acid) or RNA (ribonucleic acid) from clinical specimens' samples, amplifies target nucleic acid sequences, and detects in-situ and in real time the amplificon using an electrochemical nucleic acid probe sensor during the amplification process.

The overall biochemical reaction and the detection process includes two stages: isolation and release of target pathogen nucleic acid, and amplification of target nucleic acid and in-situ measurement of the amplicon using an electrochemical sensor.

Isolating and releasing target pathogen nucleic acid: The isolation of target pathogens and the release of nucleic acids may be achieved by any suitable method, including those known in the industry and those that may be developed in the future. Typically, the isolation step includes lysing the target pathogen and extracting the released DNA/RNA for downstream amplification. When necessary, a heating process and DNase/RNase (deoxyribonuclease/ribonuclease) inhibitors may be included in the isolation process. In general, for swab samples or isolated target pathogen samples, the samples are treated with the lysing and extraction solution at ambient temperature and then mixed to make them homogeneous so that they can be readily used for downstream amplification. For urine, saliva, or sputum samples, an additional heating process may be required during sample preparation (the lysing and extraction process).

Alternatively, the isolation and release process may be achieved by using a modified commercially available DNA/RNA lysis and extraction kit. The first step in targeting the pathogen is lysis with a lysing buffer, followed by neutralization or heating to release the target pathogen's nucleic acid. These steps help to liberate the target pathogen DNA/RNA and protect the DNA/RNA from degradation by Dnase/Rnase. Then, the target pathogen DNA/RNA is purified and concentrated with magnetic beads or columns.

Amplifying target nucleic acid and in-situ measurement of the amplicon with an electrochemical sensor: According to an aspect of the present invention, amplifying the target nucleic acid sequences and in-situ measurement of the amplification products are performed using the electrochemical DNA probe sensor in the same chamber, preferably at elevated temperatures (e.g., 37 to 90° C.). In some embodiments, a reverse transcriptase is used to convert RNA to first-strand complementary DNA (cDNA). Then the target DNA sequences are amplified with nucleic acid amplification methods using target-specific primers.

Examples of nucleic acid amplification methods include isothermal amplification, such as, without limitation, (1) Loop-mediated isothermal amplification (LAMP) (described in, for example, U.S. Pat. No. 6,410,278B1; Notomi T, Okayama H, Masubuchi H, Yonekawa T, Watanabe K, Amino N, Hase T (2000), Loop-mediated isothermal amplification of DNA, Nucleic Acids Res. 28 (12): 63e-63. doi:10.1093/nar/28.12.e63), (2) helicase-dependent amplification (HDA) (described in, for example, U.S. Pat. No. 7,829,284B2), (3) recombinase polymerase amplification (RPA) (described in, for example, U.S. Pat. No. 7,270,981B2), and (4) nucleic acid sequence-based amplification (NASBA) (described in, for example, U.S. Pat. No. 9,562,837B2; Compton, J. (1991), Nucleic acid sequence-based amplification, Nature, 350(6313), 91-92. doi: 10.1038/350091a0). Other nucleic acid amplification methods may also be used, such as polymerase chain reaction (PCR). In some embodiments of the present invention, a modified isothermal amplification process is used, as will be described in more detail later. These reactions may be generally referred to as NAAT (nucleic acid amplification tests) reactions.

According to embodiments of the present invention, preferably, the reverse transcription and nucleic acid amplification reactions are carried out in a single chamber during these processes. The components required for the reactions are provided on the cartridge and present in the reaction chamber. The reaction progression is monitored by the electrochemical nucleic acid probe sensor, which is pre-fabricated in the reaction chamber. Once amplified, the produced DNA amplicon of the target sequence is hybridized with the sensor probe in the reaction chamber. The sensor monitors the amount of amplicon formed in real time.

Detecting amplified amplicon with the electrochemical sensor: In accordance with an aspect of the present invention, amplified amplicon corresponding to target sequences are detected during the amplification process using the electrochemical sensor. In one embodiment, the amplified target DNA hybridizes to a surface-modified oligonucleotide (the probe oligonucleotide). The probe oligonucleotide is functionalized with a detectable redox reporter and attached to the electrode surface of the electrochemical sensor. If amplified target nucleic acid is present, a hybridization complex is formed on the electrode surface. The electrochemical signal will change upon this hybridization.

### Overview of the Detection System

The electrochemical nucleic acid detection system according to embodiments of the present invention is designed to permit a rapid and low-cost assay of DNA or RNA signatures indicative of the presence of a target pathogenic bacteria, virus, or organism in clinical samples. The system is rapid, sensitive, specific, and easy to use both in the clinical setting and deployed in the field. One important aspect lies in the reaction sensor cartridge (microfluidic electrochemical biosensor cartridge). The cartridge includes one or more electrochemical probe sensor units placed inside the nucleic acid amplification chamber to mitigate thermal reaction interference. The electrochemical probe sensors continuously monitor and report reaction progress in real time, enabling the semi-quantitative detection of the amplification product. Additionally, in some embodiments, the sensor unit incorporates multiple redox-labeled nucleic acid probes within a single sensor unit and/or a multi-sensor array configuration. This structure facilitates in-situ multiplex nucleic acid amplification tests (NAAT) for different targeted gene sequences.

The detection system involves three important aspects:

First, according to one aspect of the present invention, the sensor array of the system, which is electrochemical DNA probe (eDNA probe, E-DNA probe) based, is directly integrated in the NAAT reactions. By placing the eDNA probe sensor in the NAAT reaction chamber, the system can simultaneously measure the reaction produced amplicons while the reaction progresses. The NAAT reaction can be either an isothermal (e.g. Loop-mediated isothermal amplification (LAMP)) or thermal cycling (e.g. polymerase chain reaction (PCR)) amplification method, or some other NAAT method.

Second, the inventor of the present invention discovered that the eDNA probe's annealing or hybridization with the amplicons should be more favorable than the annealing or hybridization of the NAAT reaction primers with the amplicons. The inventor also discovered that the eDNA probe having a higher probe hybridization rate or higher annealing temperature than that of the primers will result in such a favorable reaction. In one embodiment, a locked nucleic acid (LNA) probe is used to achieve such a condition.

Third, the inventor also discovered that the terminal of the probe sequence phosphate backbone should be blocked by termination redox or modification chemical group for generating stable signals. If the eDNA probe is involved in the NAAT reaction primer extension process, the eDNA sensor result will carry significant non-specific reaction result. Preferably, both the 3′ and 5′ end are modified; optionally, only the 3′ end is modified.

### Structures of the Detection System

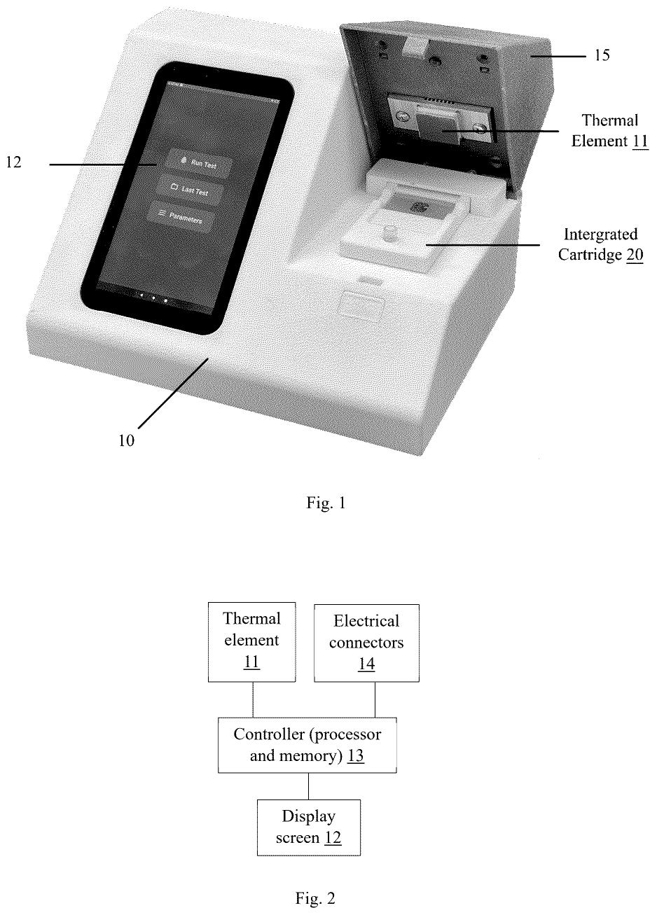

As illustrated in FIG. 1, an electrochemical nucleic acid detection system according to an embodiment of the present invention includes a multifunction electrochemical detection and control instrument 10, which is preferably a benchtop device, and a reaction sensor cartridge (a microfluidic electrochemical biosensor cartridge) 20, which is a single-use component used in conjunction with the benchtop instrument 10. The electrochemical detection and control instrument 10 manages the temperature control to perform target amplification directly on the cartridge. It also handles signal recording and processing to generate the detection result. The cartridge 20 is a microfluidic device that automates sample processing. It contains reagents necessary for the following processes: sample lysis, nucleic acid extraction, nucleic acid amplification, and real-time amplicon detection.

As shown in FIG. 2, the electrochemical detection and control instrument 10 includes a display screen 12 (and/or other user interface structures such as push buttons) disposed on an exterior of the instrument housing to provide a user interface (e.g., to receive commands from the user and to display test results to the user), a controller (computing unit) 13 disposed inside the housing, one or more optional thermal elements 11 configured to interact with the cartridge 20, and electrical connectors 14 (host electrical connectors) for electrical coupling to the cartridge 20 to transfer power and electrical signals between the cartridge and the instrument 10.

In the illustrated embodiment of FIG. 1, the detection and control instrument 10 has a pivotable portion 15 that can be lifted to accommodate the cartridge. In use, the pivotable portion 15 is lifted, the cartridge is placed on a platform below it and electrically coupled to the electrical connectors 14, and the pivotable portion is lowered so that the thermal element contacts the cartridge 20. Of course, the physical form of the detection and control instrument 10 is not limited to that shown in FIG. 1; any suitable constructions may be used. For example, in lieu of a pivotable portion 15, a slots may be provided on the housing of the instrument 10 for the cartridge 20 to be inserted into.

The controller 13 may be implemented by suitable electronic circuitry such as FPGAs (field programmable gate arrays), processors executing program code stored in memories, etc. The controller controls the thermal elements 11 and the display screen 12, and performs data acquisition, recording, processing, and display and other user interaction.

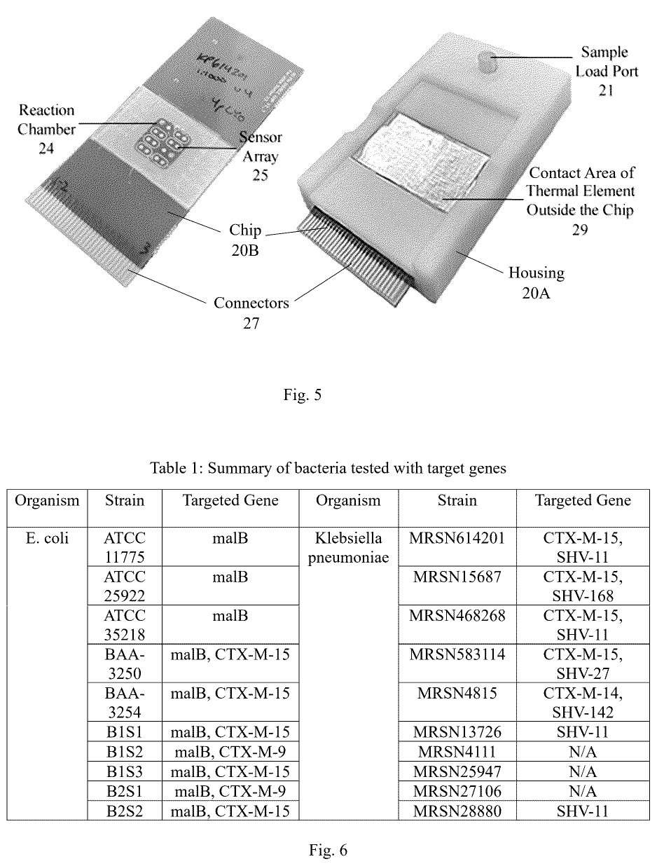

In preferred embodiments, the cartridge includes a housing 20A with a chip 20B disposed inside (see FIG. 5, where the left-hand side image shows a chip, and the right-hand side image shows a chip disposed inside the housing except for the exposed connectors). The chip 20B may be based on a printed circuit board (PCB), with the various microfluidic chambers and microchannels as well as the eDNA sensors (described below) integrated on the PCB. The chip 20B may alternatively use a glass substrate and use screen printed electrode array, or other suitable substrates and electrode structures.

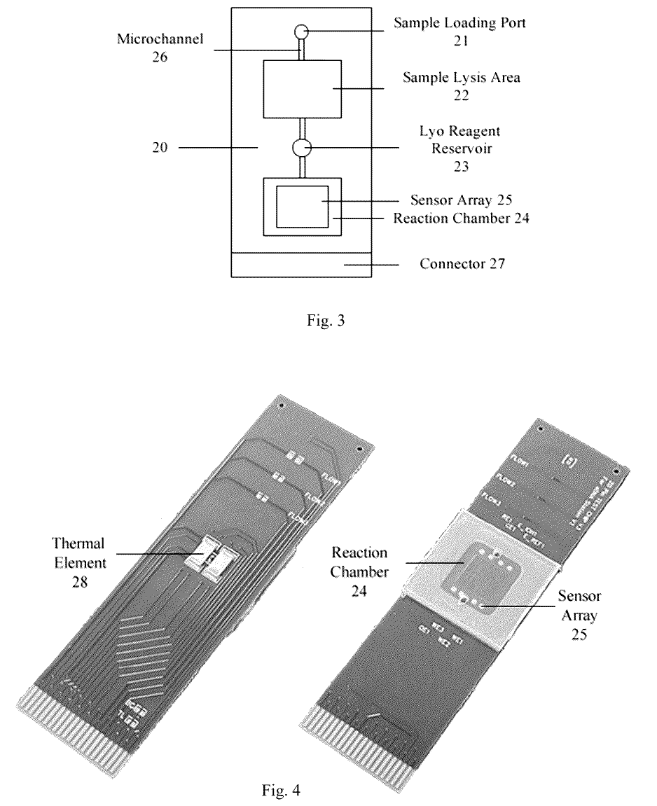

As schematically illustrated in FIG. 3, the reaction sensor cartridge 20 includes the following components:

Sample loading port 21: The entry point where the sample is introduced into the cartridge. The loaded liquid is typically the mix of a clinical sample (such as urine, whole blood, sputum, swab, stool, bacteria colony and other body fluid) and a formula buffer.

Microchannels 26: Multiple microchannels direct the sample flow through the various components 21-24 of the cartridge. The integrated microchannels ensure the sample flows sequentially through these components, facilitating automated sample processing and detection.

Sample lysis area 22: After the sample is loaded into the loading port 21 of the cartridge, the liquid flows through a microchannel to a designated area 22 where the sample undergoes lysis, breaking down cells in the sample to release nucleic acids (by heating or a chemical process or both). Depending on the lysis process used, the sample lysis area may include one or more lysis chambers for carrying out the steps of sample lysis. If a chemical process is used, reagents for sample lysis and nucleic acid extraction are provided in one of the lysis chambers. In some embodiments, this area may include a purification chamber and a concentration chamber. The sample lysis area 22 may be omitted if sample lysis is carried out off-cartridge.

Reagent reservoir 23: A small storage reservoir stores nucleic acid amplification test (NAAT) reagents needed for the nucleic acid amplification reaction. The NAAT reagents include enzyme mix, primer mix (or primers) and supplemental chemical reagents. In some embodiments, the reagents in the reservoir are lyophilized. After sample lysis, the lysis liquid flows through the reservoir 23, and rehydrates the reagents. It should be noted that in lieu of a separate reagent reservoir 23, it is also possible to provide the NAAT reagents in the NAAT reaction chamber itself.

NAAT reaction chamber 24: The liquid with the lysis sample and NAAT reagent finally flows into the NAAT reaction chamber. The NAAT reaction is continually incubated at reaction temperatures, where the temperature is controlled by one or more on-chip heating elements (described later) or the one or more heating elements 11 on the instrument 10, controlled by the instrument 10 in either case. During the reaction, the primers will hybridize with the target template or amplicon to form partial hybridized helix, and the enzyme then extent the primer from the 3′ end to generate new full double helix amplicons.

Sensor array 25: One or more sensor units are fabricated to form a sensor array 25 and integrated into the NAAT reaction chamber 24. The sensor array includes a board and multiple sensor units (see below) located on the board, forming a sensor array within the NAAT reaction chamber 24. In preferred embodiments, each sensor unit is smaller than 1 mm² in size. Placed at the bottom of the reaction chamber 24, the sensor array detects the amplified nucleic acid amplicons, and monitors the amplification process as discussed above. FIGS. 4 and 5 (described in more detail later) show two exemplary sensor arrays 25 integrated in respective reaction chambers 24.

Sensor unit: The eDNA sensor unit is a reagent-free electrochemical DNA probe-based biosensor. The DNA probe will hybridize with the targeted amplicons which results in a conformational change of the DNA probe. The DNA probe is labeled with an electrochemical redox reporter for monitoring the conformational change by measuring the change in redox electron transfer rate between the redox reporter and the electrode. These sensors may be fabricated by, for example, covalently attaching a redox reporter-labeled nucleic acid probe (eDNA probe) to a flat, conductive electrode surface. During the NAAT reaction, the eDNA probe hybridizes with the amplified targeted amplicons, and then folds into a double-helix, which changes the redox Faradaic current. The above working principle of the eDNA sensor unit is described in U.S. Pat. No. 8,003,374 . To apply the eDNA sensor in the electrochemical biosensor system described herein, however, the following improvements are required, as discovered by the inventor of the present invention.

First, in the detection process, the eDNA probe's annealing or hybridization with the amplified amplicons should be more favorable than the annealing or hybridization of the primers with the amplicons. Second, the terminal of the eDNA probe phosphate backbone should be blocked by either a termination redox or a termination chemical group to prevent the enzyme extension of the probe 3′ end for reducing the non-specific result.

This reagent-free sensor is highly effective in complex media due to its hybridization-induced current change being insensitive to non-specific adsorptions (such as enzyme and non-related DNA), ensuring accurate performance directly within the reaction.

Heating elements: The structures for heating the sample may be implemented in various ways. In a first example, shown in FIG. 4, left-hand side image (view of the back side of the chip), the thermal element (heating element) 28 is integrated directly on the chip, positioned on the backside of the sensor array and NAAT reaction chamber. The thermal element is electrically coupled to the electrical connectors 14 of benchtop instrument 10. The right-hand side image of FIG. 4 (view of the front side of the chip) shows the sensor array 25 integrated in the amplification chamber 24 on the chip. This configuration ensures precise thermal control directly at the reaction and sensor measurement site.

In another example, shown in FIG. 5, right-hand side image (view of the front side of the cartridge), the thermal element (heating element) is located within the instrument 10 and outside of the cartridge, such as the heating element 11 shown in FIG. 1. In this example, the heating element 11 directly contacts an area 29 on the cartridge (see FIG. 5 right-hand side image), providing external thermal control to the reaction chamber. Preferably, a material with high thermal conductivity (e.g., an aluminum foil) is provided in the area 29 that directly contacts the heating element 11, between the heating element and the reaction chamber.

Heating elements may also be provided on the back side of the chip at a location corresponding to the sample lysis area 22, if a heating process is used for sample lysis. Each heating element includes resistive heating elements and a built-in temperature sensor for providing controlled hearing.

Electrical connectors: A plurality of electrical connectors 27 (cartridge electrical connectors) are configured to be electrically coupled to the electrical connectors 14 of the instrument 10. The electrical connectors are electrically coupled to the sensor array 25 and the on-chip heating elements 28, and function as an interface to electrically connect the cartridge 20 to the instrument 10 for transmitting power, control signals, and electrochemical detection signals between the two.

### Operating Principle

Amplification of target nucleic acid:

The target nucleic acid is amplified with specific primers by nucleic acid amplification methods, such as isothermal amplification, modified isothermal amplification, or traditional thermal cycling amplification. During the amplification process, if the target is an RNA, the target is first reverse transcribed to the first strain cDNA. The cDNA or original DNA target are amplified to amplicon during the amplification process.

In embodiments of the present invention, the sensor array can work with either isothermal amplifications, or modified isothermal amplification, or traditional PCR-like thermal cycling amplification.

In isothermal amplification, DNA replication occurs at a constant temperature without the need for thermal cycling, unlike traditional PCR. Instead of using heat to denature DNA strands, this method relies on strand-displacement polymerases and specially designed primers to achieve strand separation and replication. The reaction temperature typically ranges between 37° C. and 75° C., depending on the specific polymerase and amplification chemistry used. Since isothermal amplification occurs at a lower temperature, it enables easy and fast DNA replication processes. Because the amplification temperature is often lower than the hybridization temperature of electrochemical sensor probes, careful probe design is essential to ensure effective binding to target amplicons. The hybridization efficiency depends on primer selection, buffer composition, and reaction conditions, which must be optimized to maintain specificity and minimize non-specific interactions.

In a modified isothermal amplification method according to some embodiments of the present invention, controlled temperature drop cycles are introduced during the amplification process to enhance annealing efficiency and improve sensor signal detection. Unlike traditional PCR, which relies on heat denaturation, isothermal amplification utilizes strand-displacement polymerase and specifically designed primers to separate and replicate DNA strands at a constant temperature. However, in the modified isothermal amplification process, periodic temperature drops, ranging from 5° C. to 20° C. below the isothermal amplification temperature (which typically ranges between 37° C. and 75° C.), are performed at periods of 3 to 15 minutes, with each cooling phase lasting 1 to 5 minutes, repeated for 1 to 40 times, preferably 5-30 times, and more preferably 10-15 times. Preferably, measurements (sensor data) are taken only during the cooling phase. The cooling phase is believed to promote stable sensor measurement due to more favorable hybridization of the eDNA probe with the amplified amplicons at the cooler temperature. The numbers of repetitions given above are not limiting and any suitable number of repetitions may be used. Generally speaking, too few repetitions will give too few data points in the time dimension; too may repetitions may lead to possible non-specific reaction due to the cooling. This controlled temperature fluctuation promotes more efficient annealing, ensuring stronger hybridization between the amplified DNA and the electrochemical sensor probes. By optimizing the annealing phase, the modified isothermal process enhances signal detection sensitivity, improving the accuracy of bacterial identification and antimicrobial resistance profiling. The temperature control is performed by the controller 13 via the heating elements provided on the cartridge 20 and/or in the benchtop instrument 10. It should be noted that the above modified isothermal amplification and detection method is not limited to devices employing electrochemical sensors, but can be more generally applied in any probe-based sensor system that is integrated with amplification reactions, such as sensor systems that employ optical detection principles. As discussed above, the cooling phase is believed to promote stable sensor measurement due to more favorable hybridization of the eDNA probe with the amplified amplicons at the cooler temperature; this principle applies to not just probe-based electrochemical sensor system but probe-based optical sensor system or other types of probe-based sensor systems.

Polymerase Chain Reaction (PCR) like thermal cycling method is a widely used nucleic acid amplification technique that relies on thermal cycling to amplify specific DNA sequences. Each PCR cycle consists of three main steps: denaturation, annealing, and extension, repeated for 20 to 40 cycles to achieve exponential DNA amplification. In the denaturation cycle, the double-stranded DNA template is heated to a high temperature, causing the hydrogen bonds between complementary strands to break, resulting in single-stranded DNA. In the annealing, the temperature is lowered to allow primers to hybridize to their complementary target sequences on the single-stranded DNA. The annealing temperature varies depending on primer properties and sequence composition. In the optional extension cycle, the DNA polymerase extends the primers by adding nucleotides to synthesize a new complementary DNA strand. Among these phases, the annealing phase is particularly favorable for electrochemical sensor detection because it provides a stable environment where single-stranded amplicons hybridize with target-specific probes. Since the temperature is lowered during annealing, binding efficiency is maximized, allowing for improved signal detection in the electrochemical system. This phase presents an optimal window for monitoring nucleic acid hybridization events, as the sensor can detect the increasing concentration of amplified DNA with high specificity. The electrochemical detection system according to some embodiments of the present invention leverages this principle by aligning the electrochemical detection timing with the annealing phase, enhancing real-time signal acquisition and improving sensitivity in multiplex pathogen detection.

In addition to temperature optimization, the primer design, buffer composition, and ion-selective monitoring conditions should be fine-tuned to achieve high specificity and amplification efficiency. Buffer salts, their concentrations, and pH levels can play important roles in reaction stability and sensor performance. Furthermore, denaturation reagents such as urea, dimethyl sulfoxide (DMSO), and guanidine chloride can be incorporated to accelerate amplification while maintaining signal integrity.

The amplification process according to embodiments of the present invention can amplify one target sequence or multiple target sequences in one reaction. When multiple primer sets are added to the reaction, multiple target sequences can be amplified at once, although the reaction time and temperature, and primer sets concentration need to be optimized. When multiple targets are amplified together, it can increase the detection efficiency, sensitivity, and specificity.

Primer design is one of the key factors that affect the amplification sensitivity and specificity. Well-designed primers should specifically target the sequence while achieving high amplification efficiency. Several key factors need to be considered, such as melting temperature, stability at the end of each primer, and secondary structure. Each amplification has its specified primer design guideline. Methods and tools for the design of the oligonucleotide primers are well studied. As an example, for LAMP amplification, various software tools are widely available to assist in primer design. The most commonly used software for LAMP primer design is Primer Explorer. It is easy-to-use and free. Another web-based software is the NEB LAMP primer design tool, which is also free and very similar to Primer Explorer. The user interface is easy and friendly. Several alternative software is available for LAMP primer designing, including LAVA, LAMP Designer, STITCHER, FastPCR, and GLAPD. For other isothermal amplification such as RPA, HAD, NASBA, et al., there are several design software and optimization tools available as well, including IDT Primer Quest, Applied Biosystem Primer Express™, Genescript design tool. Several variables are important for the primer design, including the primer length and Tm parameters, and potential secondary structure, etc. Another important variable is the potential cross-activity with other genome sequences. It is important to run BLAST analysis against public domain nucleotide sequences in NCBI (National Center for Biotechnology Information) nucleotide collection to validate that there is no cross activity.

In-situ detecting the amplicon with an electrochemical nucleic acid probe sensor:

In the nucleic acid amplification method according to embodiments of the present invention, the target nucleic acid is amplified with specific primers (NAAT reaction) to produce DNA amplicon. In order to detect the targeted amplicon product in real time, the electrochemical nucleic acid probe sensor array is directly integrated in the NAAT reaction. By placing the eDNA probe sensor in the NAAT reaction chamber, the device can simultaneously measure the reaction-produced amplicons while the reaction progresses. The NAAT can be either an isothermal amplification method (e.g. LAMP), or a modified isothermal amplification method, or thermal cycling (e.g. PCR) amplification method, or some other NAAT method.

According to embodiments of the present invention, the eDNA probe is designed to have a higher probe hybridization rate or higher annealing temperature than the annealing or hybridization of the primers with the amplicons, which will result in a favorable reaction. In one embodiment, an LNA probe is used. During the NAAT reaction, eDNA probe annealing or hybridization with the amplicons must be more favorable than the annealing or hybridization of the NAAT reaction primers with the amplicons. During the NAAT reaction, the probe hybridizes with the amplicon and produces the probe conformation change. The electrochemical nucleic acid sensor array is capable of monitoring the real-time reaction as the amplicon accumulates.

Probe oligonucleotides are synthesized with suitable modifications to allow efficient linkage to the electrode surface and are modified with an electrochemical redox reporter for measuring the hybridization response. The probe oligonucleotide is designed to have a complementary sequence that is complementary to the converted target nucleic acid at a region. The length of the complementary sequence is typically approximately 15 to 45 bases in length. Several different probe oligonucleotides may be used in the multiplex condition. In some cases, it may include a spacer sequence (e.g. 1 to 10 T bases) at the end of the oligonucleotide attached to the surface to increase the probe flexibility. In some cases, the probe oligonucleotide may include a double-stranded oligonucleotide near the surface to form a hairpin structure to reduce the background signal and non-specific signal. Probe oligonucleotides can be synthesized using DNA. The incorporation of modified nucleic acids such as PNA (peptide nucleic acid) or LNA (locked nucleic acid) may be useful for the enhanced hybridization properties of the target DNA and reduces non-specific responses.

### Electrochemical Nucleic Acid Sensor Fabrication

The electrochemical sensors may be fabricated based on known methods of fabricating electrochemical nucleic acid sensors, with appropriate modification for the attachment to the electrode. For example, sensors may be prepared using methods similar to previously described sensors, for example, as described in Kang et al. “Comparing the properties of electrochemical-based DNA sensors employing different redox tags” Analytical chemistry 81 (21), 9109-9113; Kang et al. “Survey of redox-active moieties for application in multiplexed electrochemical biosensors”, Analytical chemistry 88 (21), 10452-10458; U.S. Pat. No. 8,003,374 by Heeger, Fan, and Plaxco; Rowe et al. “Fabrication of electrochemical-DNA biosensors for the reagentless detection of nucleic acids, proteins, and small molecules,” Journal of visualized experiments: JoVE, 52 2922. 1 Jun. 2011. The detailed fabrication protocol is described below.

Redox reporter conjugated self-assembly nucleic acid probe (probe oligonucleotides): The nucleic acid probes are double-end-modified single-stranded oligonucleotides. The single-stranded oligonucleotide is designed to provide a highly efficient signal response with less non-specified signals. The probe oligonucleotide is selected to avoid any cross-activity with other common public genes, which can cause a non-specific response. One end of the single-stranded oligonucleotide is modified with an anchor element to attach it to the electrode surface, for example, by covalent bonding to the electrode or to a monolayer on the electrode, or via chemisorption or adsorption. Anchoring moieties may include elements that form self-assembled monolayers on the electrode surface. Such monolayers may be generated on metal surfaces, such as Au, Ag, and Cu, using the thiol chemistry, or alkylsiloxanes on hydroxyl-terminated surfaces, such as Si/SiO2, Al/Al2O3, glass surfaces, using siloxy linkages, etc.

Another end of the single-stranded oligonucleotide is functionalized with one or more electrochemical redox reporters. The redox reporter is any composition of matter that interacts with the selected electrode material creating a Faradaic current. The redox reporter may conjugate to the probe by any appropriate chemistry. For example, as described in Kang et al. “Comparing the properties of electrochemical-based DNA sensors employing different redox tags,” Analytical chemistry 81 (21), 9109-9113 and Kang et al. “Survey of redox-active moieties for application in multiplexed electrochemical biosensors”, Analytical chemistry 88 (21), 10452-10458, the redox reporter may be conjugated to the amine-modified single-stranded DNA via NHS-ester amine reaction. Amine modified DNA aqueous solution may be mixed with redox reporter-NHS ester in dry dimethyl sulfoxide (DMSO) at room temperature. The sequence design and redox reporter design and selection are important during the detection process, since the oligonucleotide-modified probe sensor is soaked in a protein-rich reaction solution, which condition is challenging to the redox reporter stability.

Fabrication of redox reporter-modified electrochemical DNA sensor: In one embodiment, the electrochemical nucleic acid sensors are prepared with a gold electrode. The gold electrode surface is cleaned before immobilizing with a probe oligonucleotide. The redox reporter-modified probe oligonucleotide is attached to the electrode surface, for example, via thiol-gold self-assembly. The electrode surface is passive, with a continuous monolayer of small molecules to prevent non-specific absorption in protein-rich conditions. The passive continuous monolayer of small molecules can be self-assembled after the probe oligonucleotide is attached to the surface, or the small molecules can be mixed with the probe oligonucleotide at a defined ratio and both the small molecules and the probe oligonucleotide are assembled simultaneously. This sensor can be stored dry or wet for a long period.

Redox reporter-modified electrochemical DNA sensor characterization: Electrochemical nucleic acid sensors can be characterized with electrochemistry voltammetry, examples of which include, without limitation, square wave voltammetry, cyclic voltammetry, differential pulse voltammetry, alternating current voltammetry, potentiometry, or chronoamperometry. In one embodiment, the use of kinetic differential measurement techniques, as known in the art, can be employed to improve the signal-to-noise ratio. The measured current directly indicates the coverage of surface nucleic acid probes. The sensor produces almost no signal response to the environmental change, such as proteins and non-specific oligonucleotides. During the test, once the surface probes hybridized with complementary oligonucleotides, the measured signal will show a significant decrease.

### EXAMPLES

### Amplification and Semi-Quantitative Detection of e coli and ESBL Resistance Gene Using LAMP and Electrochemical DNA Probe Sensor

These examples demonstrate the integration of LAMP amplification and electrochemical nucleic acid platform for e. coli and ESBL resistance gene detection. This bacteria application is used as a model target to show the sensitivity, specificity, semi-quantitative, and multiplexing of this detection system.

### Materials and Methods:

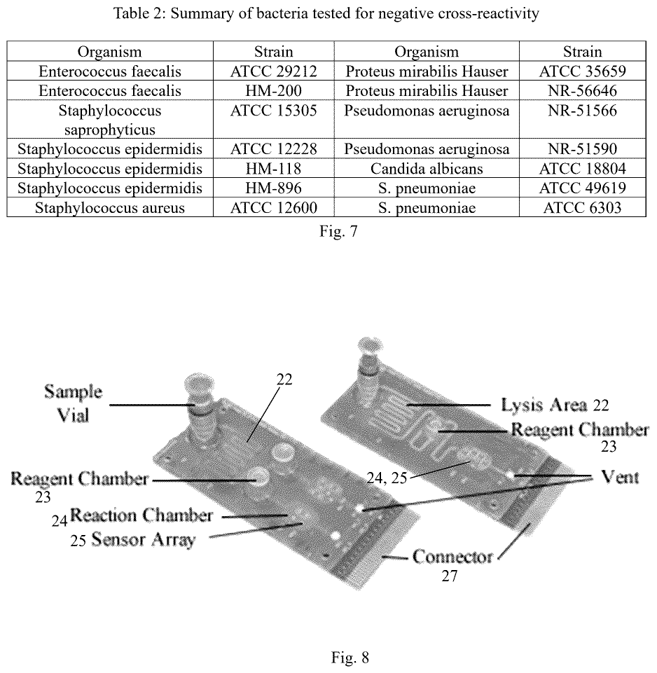

Materials: The bacteria used for positive controls, negative controls, cross-reactivity tests were purchased from ATCC and collected from clinical samples. Both single plex and multiplex reactions were tested with a large selection of different bacteria strains (see Table 1 in FIG. 6). Through cross reaction analysis, it was confirmed that the primers were specific to the target genes and did not show cross-reactivity with other bacterial flora (see Table 2 in FIG. 7). The LAMP reaction mix used WarmStart LAMP Kit (New England Biolabs) and Meridian Lyo-Ready LAMP Urine Master Mixes, and the PCR reaction used Luna Universal qPCR & RT-qPCR kit (New England Biolabs). All LAMP primers and PCR primers were custom design oligonucleotides purchased from IDT (Integrated DNA Technologies).

Conjugating the redox reporters to the DNA probes: The redox reporters were conjugated to the probe single-stranded oligonucleotide via the NHS-ester reaction. The redox reporter conjugated with the 5′-amine modified single-stranded DNA (purchase from Integrated DNA Technology). 10 μl of 200 μM 5′ amine-modified DNA aquae solution was added to 50 μl of a 0.5 M sodium bicarbonate buffer (pH 8.5). 1 μmole of methylene blue-NHS ester (purchased from ATTO Bio) was dissolved in 10 μl dry dimethyl sulfoxide (DMSO). The DNA aquae solution was mixed with the methylene blue-NHS ester DMSO solution, and the mixture was incubated for 4 hours in dark at room temperature. After the reaction was completed, the crude product was desalted using PD-10 desalting columns (Cytiva Life Sciences) and purified by RP-HPLC (Agilent). The purified DNA solutions were lyophilized and stored at −20° C. for future use.

Fabrication of gold electrode: The sensor electrode array was custom designed and fabricated gold-plated printed circuit board (PCB) electrode array. The PCB electrode was covered with 2 μm pure soft gold. Ag/AgCl was deposited via a screen-printing process with Ag/AgCl ink.

Fabrication of redox DNA modified electrochemical sensor: The gold electrode was scanned with cyclic voltammetry in 0.5M sulfuric acid 20 times to clean and activate the gold surface. The redox reporter modified thiol DNA was reduced for 1 hr at room temperature in 10 mM tris (2-carboxyethyl) phosphine hydrochloride (Sigma Aldrich) and then diluted to a final concentration of 100 nM in PBS buffer (pH 7.4). The gold electrodes were incubated in this solution for 1 hr at room temperature, then rinsed with deionized water. After probe DNA deposition, the electrode surface was “backfilled” with 3 mM Mercaptohexanol aqueous solution for 120 min to form a continuous self-assembled monolayer. Following this, the electrodes were rinsed in deionized water and stored in the buffer at 4° C. for future use.

Fabrication of eDNA cartridge: The prototype microfluidic cartridges (single and double chamber) were capable of processing urine samples, performing on-chip lysis, conducting in-situ LAMP amplification and real-time analysis with integrated eDNA sensors. The eDNA sensor array was located in the LAMP reaction chamber. The cartridges were optimized for a 50 μL reaction volume and refined for sample loading, multi-chamber amplification, and controlled fluid flow using a pressure-sense feedback-control system. A temperature-controlled heating unit was incorporated on the cartridge under the lysis channel, while a separate heating and cooling system for LAMP assay thermal control was provided on the benchtop instrument 10. This approach expanded the sensor array area while simplifying the chip design, effectively reducing manufacturing complexity and cost. Both the lysis and LAMP temperature controls were managed by the benchtop instrument 10.

FIG. 8 illustrates two exemplary microfluidic sensor cartridges for the electrochemical nucleic acid detection system. The cartridge shown on the left-hand side is a two-chamber cartridge, in which the lysis area 22 feeds two reagent chambers 23, and each reagent chamber is associated with a downstream reaction chamber 24 with a sensor array 25, and a vent further downstream. The cartridge shown on the right-hand side is a single-chamber cartridge having a single set of lysis area 22, reagent chamber 23, reaction chamber 24 with sensor array 25, and vent. In addition, both cartridges feature a sample loading port, a sample vial, and connectors 27.

Multiplexed amplification reactions: LAMP reaction was set up using Warmstart RT-LAMP kit. One single reaction was 25 μL, containing 12.5 uL 2X Warmstart stock master mix, 2.5 uL 10X Primer mix containing six LAMP primers (B3, F3, LB, BIP, FIP), 4.8uL nuclease-free water, and 5 uL sample extraction solution. The 10X primer mix solution was prepared with 0.2 uM F3 & B3, 0.4 uM LB & LF and 1.6 uM FIB & BIP. The reaction was continually incubated at alternating 63° C. for 5 min and 45° C. for 1 min as one cycle, and repeated 12 cycles on the heating block.

The multiplex LAMP reaction solution was performed with the same steps as a single plex reaction, except when preparing the master mix multiple sets amplification primers (E coli housekeeping gene and ESBL antibiotic resistant genes) were added to the master mix.

Redox DNA modified electrochemical sensor characterization: The electrochemical nucleic acid sensors were characterized with square wave voltammetry (SWV) with a 50 mV amplitude signal at 50 Hz frequency scanning from −500 mV to 0 mV during the reaction. The signal change was calculated by the relative change in SWV peak currents when the reaction started. The peak current was measured every 5 mins.

Results:

The eDNA probes in the examples were designed to detect specific LAMP amplicons in real-time by hybridizing with the loop regions of the LAMP product. Each eDNA probe was designed to complement regions between B2 and B1c (near LB) during the reaction. Modified nucleic acid was incorporated to further stabilize the probes and optimize the electrochemical redox position which enhanced the detection sensitivity. To further stabilize measured signals, thermostable trihexylthiol anchors were used to immobilize the probe to the gold surface to improve the stability of the attachment under higher temperatures.

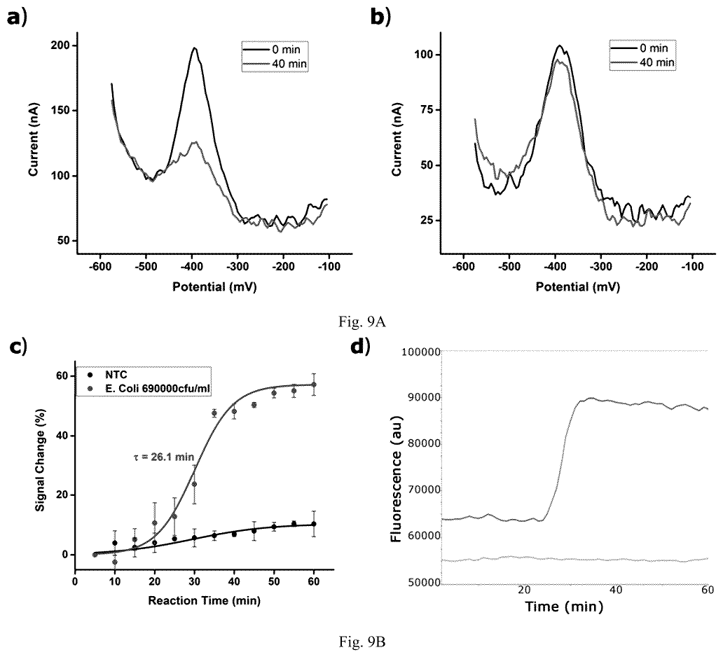

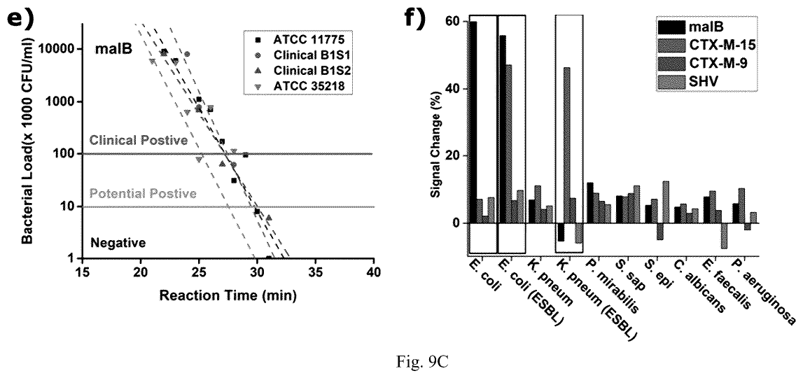

The results are shown in FIGS. 9A, 9B, 9C. FIG. 9A shows the hybridization complex concentration as represented by the relationship of the redox Faradaic current vs. the electrical potential applied to the electrode. Comparison of the two current vs potential curves at 0 minute and 40 minutes shows a reduction of the current in diagram a). More specifically, target-specific capture probes were immobilized on gold electrodes within the eDNA microarray on the cartridge. The amplified target DNA hybridized to the capture probe and to a complementary methylene blue-labeled signal probe. Electrochemical analysis determined the presence or absence of targets using voltammetry during the reaction. Upon target hybridization, the redox signal decreased, which was measured using square wave voltammetry (SWV). The X-axis represents the electrochemical potential referenced against Ag/AgCl, while the Y-axis represents the redox current. Diagram b) shows that the probe signal (the current vs. potential curve) showed no response when the amplified target was not specific to the probe.

In FIG. 9B, diagram c) shows the in situ eDNA sensor monitored LAMP amplification of the E. coli malB gene, with signal changes correlated to reaction progression (reaction time). In this diagram, the upper curve represents the E. coli sample, where the signal began to rise rapidly after 20 minutes, indicating amplification, and reached completion at around 30 minutes. The lower curve represents the no-template control (NTC) sample, showing no signal change throughout the reaction, confirming specificity. The same reaction under identical conditions was measured using traditional fluorescence detection, and the result is shown in diagram d), demonstrating a strong correlation between fluorescence and eDNA sensor results, validating the electrochemical approach.

In FIG. 9C, diagram e) show that bacterial load exhibited a linear correlation with reaction time in a semi-log plot using urine samples, demonstrating the biosensor's quantitative capability. The diagram shows four different e coil strains. Diagram f) shows a 4-plex assay which was able to specifically detect target ESBL genes (malB, CTX-M-1, CTX-M-9, SHV) without cross-reactivity to non-target bacterial flora or common uropathogens, confirming high specificity of the biosensor.

After exploring multiple primers, and then multiple premier sets (combinations for multiplexing) with the goal of reducing interference between primers, a number of LAMP primers were selected and combined with eDNA probes into a multiplex assay capable of identifying multiple ESBL genes and E. coli from urine samples. Semi-quantitative measurement for bacterial load across different strains was successfully achieved, confirming the robustness of the assay. The linear relationship between reaction time and bacterial load (see FIG. 9C, diagram e) was demonstrated using the optimized master mix formulation, as validated through parallel bacterial culture counts. The linear detection range was between 10,000 to 10,000,000 CFU/ml, which effectively covers the clinical range for urine samples (over 50% signal suppression for positive samples) and low background noise (less than 10%) (FIG. 9C, diagram e). As shown in FIG. 9C, diagram e, quantification using serial dilutions of E. coli demonstrated reliable detection, with 10,000 CFU/ml yielding an approximate reaction starting time (Ct) value of 30 minutes. (Note that CFU is a measure of the quantity of bacteria, although in practice, what is actually measured is the quantity of the target bacteria DNA; because it is possible that some bacteria may carry more sets of the target DNA, the vertical axis in FIG. 9C may be alternatively expressed as the quantity of bacteria NDA.) The eDNA sensor's output was calibrated to a standard curve, bacterial load was linear regressed to reaction time in a semi-log plot (FIG. 9C, diagram e). This allows the system to provide semi-quantitative bacterial counts. Importantly, the multiplex assay-maintained sensitivity was comparable to monoplex reactions, with no loss in performance. Specificity testing of the target genes using DNA mixtures of three strains, differing by <100 in concentration, demonstrated 100% accuracy in a panel of 10 clinical isolates of E. coli.This confirmed the assay's capacity for accurate, semi-quantitative bacterial detection in clinical settings.

In a cross-reactivity analysis of the 4-plex assay, it was confirmed that the results were highly specific to the target genes. No cross-reactivity was observed with other bacterial flora or common uropathogens present in the clinical samples tested. This specificity is critical for ensuring accurate diagnostics, particularly in multiplex reactions, where the detection of multiple targets simultaneously must not be compromised by interactions with non-target organisms. These results validated the reliability of the assay in differentiating between ESBL-related genes and other bacterial species commonly found in urinary tract infections.

It will be apparent to those skilled in the art that various modification and variations can be made in the semi-quantitative electrochemical biosensor system and method of the present invention without departing from the spirit or scope of the invention. Thus, it is intended that the present invention cover modifications and variations that come within the scope of the appended claims and their equivalents.

1. A microfluidic electrochemical biosensor cartridge comprising:
a sample loading port;
a reagent reservoir containing nucleic acid amplification test (NAAT) reagents, including a target-specific NAAT reaction primer;
a nucleic acid amplification reaction chamber (NAAT reaction chamber);
an electrochemical sensor array disposed within the NAAT reaction chamber, including an electrochemical sensor configured to detect an amplified target nucleic acid sequence, the electrochemical sensor including an electrode and a plurality of target-specific probe oligonucleotides attached to a surface of the electrode, wherein each probe oligonucleotide is labeled with an electrochemical redox reporter and includes a complementary sequence that is complementary to the target nucleic acid sequence, and configured to undergo conformation changes upon hybridization with the target nucleic acid sequence to change a redox electron transfer rate between the redox reporter and the electrode;
a plurality of microchannels fluidicly connecting the sample loading port to the reagent reservoir and fluidicly connecting the reagent reservoir to the NAAT reaction chamber; and
a plurality of cartridge electrical connectors electrically coupled to the electrochemical sensor array.

2. The microfluidic electrochemical biosensor cartridge of claim 1, further comprising:
a substrate, wherein the reagent reservoir, the NAAT reaction chamber, the electrochemical sensor array, and the plurality of microchannels are formed on a first side of the substrate; and
a cartridge housing, wherein the substrate is disposed at least partially within the cartridge housing.

3. The microfluidic electrochemical biosensor cartridge of claim 2, further comprising a sample lysis area disposed on the first side of the substrate, the sample lysis area including one or more sample lysis chambers containing reagents for sample lysis and nucleic acid extraction,
wherein the plurality of microchannels fluidicly connect the sample loading port to the sample lysis area and fluidicly connect the sample lysis area to the reagent reservoir.

4. The microfluidic electrochemical biosensor cartridge of claim 3, further comprising:
one or both of: a first heating element formed on an opposite side of the substrate at a location corresponding to the NAAT reaction chamber and configured to heat the NAAT reaction chamber, and a second heating element formed on the opposite side of the substrate at a location corresponding to the sample lysis area and configured to heat the sample lysis area; and
a second plurality of cartridge electrical connectors electrically coupled to the one or both of the first heating element and the second heating element.

5. The microfluidic electrochemical biosensor cartridge of claim 1, wherein the reagent reservoir further contains a reverse transcriptase, and wherein the reagents in the reagent reservoir are lyophilized.

6. The microfluidic electrochemical biosensor cartridge of claim 1, wherein the probe oligonucleotides have a hybridization rate or an annealing temperature with the target nucleic acid sequence that is higher than a hybridization rate or an annealing temperature of the NAAT reaction primer with the target nucleic acid sequence.

7. The microfluidic electrochemical biosensor cartridge of claim 6, wherein the probe oligonucleotides incorporate locked nucleic acids (LNA) or peptide nucleic acids (PNA).

8. The microfluidic electrochemical biosensor cartridge of claim 1, wherein the probe oligonucleotides are single-stranded oligonucleotides, and wherein a 3′ end of each oligonucleotide is modified with the redox reporter or a termination chemical group to prevent extension of the 3′ end.

9. The microfluidic electrochemical biosensor cartridge of claim 1, wherein the probe oligonucleotides are single-stranded oligonucleotides, wherein one end of the oligonucleotides is modified with the redox reporter, and another end of the oligonucleotides is modified with an anchor moieties to attach the oligonucleotides to the electrode surface, by covalent bonding to the electrode, or by forming self-assembled monolayers on the electrode surface, or by chemisorption or adsorption.

10. The microfluidic electrochemical biosensor cartridge of claim 1, wherein the reagent reservoir contains a plurality of target-specific NAAT reaction primers respectively specific to a plurality of target nucleic acid sequences, and the electrochemical sensor array includes a plurality of electrochemical sensors respectively configured to detect amplified ones of the plurality of target nucleic acid sequences.

11. An electrochemical biosensor system comprising:
the microfluidic electrochemical biosensor cartridge of claim 1; and
an electrochemical detection and control instrument, configured to receive the biosensor cartridge, wherein the instrument includes:
a plurality of host electrical connectors configured to be electrically coupled to the plurality of cartridge electrical connectors of the biosensor cartridge;
a controller electrically coupled to the plurality of host electrical connectors, configured (a) to transmit and receive electrical signals to and from the biosensor cartridge, including to receive an electrical signal representing the redox electron transfer rate from the biosensor cartridge, and (b) to record and process the electrical signals received from the electrochemical sensor to generate a detection result; and
a user interface on an exterior of a housing of the benchtop instrument, electrically coupled to the controller.

12. An electrochemical biosensor system comprising:
the microfluidic electrochemical biosensor cartridge of claim 2; and
an electrochemical detection and control instrument, configured to receive the biosensor cartridge, wherein the instrument includes:
a plurality of host electrical connectors configured to be electrically coupled to the plurality of cartridge electrical connectors of the biosensor cartridge;
a controller electrically coupled to the plurality of host electrical connectors, configured (a) to transmit and receive electrical signals to and from the biosensor cartridge, including to receive an electrical signal representing the redox electron transfer rate from the biosensor cartridge, and (b) to record and process the electrical signals received from the electrochemical sensor to generate a detection result; and
a user interface on an exterior of a housing of the benchtop instrument, electrically coupled to the controller;
wherein either: the biosensor cartridge further comprises a first heating element formed on an opposite side of the substrate at a location corresponding to the NAAT reaction chamber to heat the NAAT reaction chamber, or: the electrochemical detection and control instrument includes a second heating element configured to contact an area of the biosensor cartridge corresponding to the NAAT reaction chamber to heat the NAAT reaction chamber.

13. The electrochemical biosensor system of claim 12:
wherein the controller is programed to control the first heating element or the second heating element to maintain a temperature of the NAAT reaction chamber at a constant temperature.

14. The electrochemical biosensor system of claim 12:
wherein the controller is programed to control the first heating element or the second heating element to: (a) maintain a temperature of the NAAT reaction chamber at a first temperature between 37° C. and 75° C. for a first time duration, (b) then lower the temperature of the NAAT reaction chamber to a second temperature which is 5° C. to 20° C. below the first temperature for a second time duration, the second time duration being 1 to 5 minutes, wherein a cycle including the first time duration followed by the second time duration is 3 to 15 minutes, and (c) repeat the cycle for 1 to 40 times.

15. A method for detecting a target nucleic acid sequence, comprising:
mixing a sample with nucleic acid amplification test (NAAT) reagents, the NAAT reagents including a target-specific NAAT reaction primer;
providing the sample mixed with the NAAT reagents to a nucleic acid amplification reaction chamber (NAAT reaction chamber),
wherein the NAAT reaction chamber is provided with an electrochemical sensor array disposed therein, including an electrochemical sensor configured to detect amplified ones of the target nucleic acid sequence, the electrochemical sensor including an electrode and a plurality of target-specific probe oligonucleotides attached to a surface of the electrode, wherein each probe oligonucleotide is labeled with an electrochemical redox reporter and includes a complementary sequence that is complementary to the target nucleic acid sequence, and configured to undergo conformation changes upon hybridization with the target nucleic acid sequence to change a redox electron transfer rate between the redox reporter and the electrode;
while the NAAT reagents react with the sample to amplify any target nucleic acid sequence in the sample within the NAAT reaction chamber, monitoring an electrical signal generated by the electrochemical sensor array which represents the redox electron transfer rate; and
determine whether the target nucleic acid sequence is present in the sample based on the monitored electrical signal.

16. The method of claim 15, further comprising:
while the NAAT reagents react with the sample to amplify any target nucleic acid sequence in the sample within the NAAT reaction chamber, controlling a temperature of the NAAT reaction chamber, including: (a) maintaining a temperature of the NAAT reaction chamber at a first temperature between 37° C. and 75° C. for a first time duration, (b) then lowering the temperature of the NAAT reaction chamber to a second temperature which is 5° C. to 20° C. below the first temperature for a second time duration, the second time duration being 1 to 5 minutes, wherein a cycle including the first time duration followed by the second time duration is 3 to 15 minutes, and (c) repeating the cycle for 1 to 40 times.

17. The method of claim 15, wherein the probe oligonucleotides have a hybridization rate or an annealing temperature with the target nucleic acid sequence that is higher than a hybridization rate or an annealing temperature of the NAAT reaction primer with the target nucleic acid sequence.

18. The method of claim 17, wherein the probe oligonucleotides incorporate locked nucleic acids (LNA) or peptide nucleic acids (PNA).

19. The method of claim 15, wherein the probe oligonucleotides are single-stranded oligonucleotides, and wherein a 3′ end of each oligonucleotide is modified with the redox reporter or a termination chemical group to prevent extension of the 3′ end.

20. The method of claim 15, wherein the probe oligonucleotides are single-stranded oligonucleotides, wherein one end of the oligonucleotides is modified with the redox reporter, and another end of the oligonucleotides is modified with an anchor moieties to attach the oligonucleotides to the electrode surface, by covalent bonding to the electrode, or by forming self-assembled monolayers on the electrode surface, or by chemisorption or adsorption.
